(19) United States
(12) Patent Application Publication
(10) Pub. No.: US 2010/0234469 A1
(43) Pub. Date: Sep. 16, 2010

(54) New Crystals Of A Benzoylbenzeneacetamide Derivative

(75) Inventors: Ana Gavaldá Escudé, Sant Cugat del Valles (ES); Ernesto Duran Lopez, Castellbisbal (ES)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Medichem, S.A., Sant Joan Despi (ES)

(21) Appl. No.: 12/723,325

(22) Filed: Mar. 12, 2010

Related U.S. Application Data
(60) Provisional application No. 61/159,697, filed on Mar. 12, 2009.

(51) Int. Cl. A61K 31/165; C07C 237/20; A61P 29/00; B32B 5/16

(52) U.S. Cl. 514/619; 564/163; 428/402

(57) ABSTRACT

The invention relates to 2-amino-3-benzoylbenzeneacetamide, i.e. nepafenac, crystals having reduced chargeability, to processes for the preparation thereof, and to the use thereof for preparing pharmaceutical formulations.

### CROSS-REFERENCE TO RELATED APPLICATION

This patent application claims the benefit of U.S. Provisional Patent Application No. 61/159,697, filed Mar. 12, 2009, which is incorporated by reference.

### BACKGROUND OF THE INVENTION

Nepafenac (compound I) is the international common accepted name for 2-amino-3-benzoylbenzeneacetamide, and has an empirical formula of C₁₅H₁₄N₂O₂, and a molecular weight of 254.28.

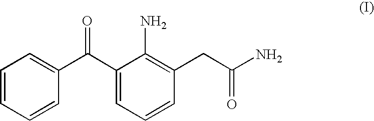

Nepafenac is a non-steroidal anti-inflammatory active pharmaceutical substance with analgesic activity. In the United States, nepafenac is marketed under the name Nevanac™, and is formulated as a suspension and indicated for ophthalmic use.

The preparation of nepafenac and similar compounds is disclosed in U.S. Pat. No. 4,313,949. In particular, Example 2 of U.S. Pat. No. 4,313,949 describes the synthesis of nepafenac which is isolated in the form of yellow needles after crystallization from isopropanol.

However, it is known that crystals in needle-like shape are of high electrostatic nature, which causes processability problems, i.e., sticking due to static electricity, less compaction, filtration difficulties, etc. Also, when preparing a solid pharmaceutical composition, crystals of such electrostatic nature are not only difficult to handle, but also involve a serious danger, which hence require the use of special safety measures. In particular, the present inventors have carried out a crystallization from isopropanol as described in Example 2 of U.S. Pat. No. 4,313,949 (see Comparative Example 1 of the present invention), and the yellow needles of nepafenac obtained therein have been proved to be of high chargeability, i.e. they have a high tendency to store electrostatic charges.

Also, it is known that crystals in needle-like shape are undesirable because, for example, filtration of suspensions of such needle-like crystals it is known to be difficult, and that bulk material comprising such needle-like crystals can be subject to blocking or bridging in weighing, handling, and conveying equipment.

Thus, in view of the foregoing there is a need to provide crystals of nepafenac with reduced or minimum chargeability.

In addition, it is known that nepafenac is practically insoluble in water, and for that reason, the drug is formulated as a suspension applied by the topical ocular route. Thus, there is also a need to provide crystals of nepafenac which might be suitable for preparing a suspension of nepafenac for ophthalmic use.

### BRIEF DESCRIPTION OF THE DRAWINGS

FIG. 1 depicts nepafenac crystals with needle-like shape as obtained under crystallization from 2-propanol.

FIG. 2 depicts nepafenac crystals with needle-like shape as obtained under crystallization from 2-propanol.

FIG. 3 depicts nepafenac crystals with needle-like shape as obtained under crystallization from 2-propanol.

FIG. 4 depicts nepafenac crystals with plate-like shape as obtained under crystallization from a mixture of 2-propanol:water 90:10.

FIG. 5 depicts nepafenac crystals with plate-like shape as obtained under crystallization from a mixture of 2-propanol:water 90:10.

FIG. 6 depicts nepafenac crystals with plate-like shape as obtained under crystallization from a mixture of 2-propanol:water 90:10.

FIG. 7 depicts the solubility profile (mg/mL) of nepafenac in 2-propanol and in different mixtures of 2-propanol with up to 40% of water, at reflux temperature.

FIG. 8 depicts nepafenac crystals with small size as obtained after micronization of nepafenac crystals with plate-like shape.

FIG. 9 depicts the Powder X-ray diffraction plots of nepafenac with small particle size obtained by spray-drying (as described in Example 10) and by micronization (similarly as described in Example 8).

### BRIEF SUMMARY OF THE INVENTION

The invention relates to 2-amino-3-benzoylbenzeneacetamide, i.e. nepafenac, crystals having reduced chargeability, to processes for the preparation thereof, and to the use thereof for preparing pharmaceutical formulations.

In particular, the invention relates to nepafenac crystals having plate-like shape which have a reduced specific surface area and hence a reduced chargeability, and to processes for the preparation thereof. Also, the invention relates to crystals of nepafenac having small size as obtained from reducing the particle size of the nepafenac crystals with plate-like shape of the invention, which show improved flowability properties (i.e. improved Hausner ratio) over crystals of nepafenac having small size obtained by reducing the particle size of the nepafenac crystals with needle-like shape.

Also, the invention provides crystals of nepafenac having small size with improved properties (i.e. homogeneous particle shape, improved sphericity, improved flowability, reduced abrasive properties for ophthalmic use, improved particle size, and improved crystallinity), characterized in that said crystals have been obtained by mechanical comminution (i.e. any conventional mechanical process for reducing the size of particles).

### DETAILED DESCRIPTION OF THE INVENTION

In an aspect, the present invention relates to nepafenac crystals having plate-like shape. It has been observed that the nepafenac crystals with plate-like shape of the invention exhibit a reduced specific surface area and hence a reduced chargeability.

The crystals of the present invention having plate-like shape are clearly distinguished from the crystals with needle-like shape obtained by the prior art processes by means of their “aspect ratio”. The “aspect ratio” of a crystal is defined as the ratio of its longest dimension to its shortest dimension. As used herein, “aspect ratio” is the quotient of the division of a crystal's length by its width. The aspect ratio of crystals can be obtained by taking micrographs of a batch of crystal (See General Experimental Conditions. Optical Microscopy). The needle-like shape crystals of nepafenac obtained by the prior art processes show an aspect ratio higher than 5.6. In turn, the plate-like shape crystals of nepafenac of the present invention have been found to show an aspect ratio of approximately 5.6 or below, preferably of 5.0 or below, more preferably of 4.0 or below, even more preferably of approximately 3.6 or below, and yet even more preferably of 3.1 or below.

Drug substances in solid form can suffer electrostatic charging by contact or friction electrification (tribocharging) caused by interactions among particles or between particles and the surfaces that contain them. These interactions can affect formulation, manufacture, powder flow, and packing behaviour. In addition, it has been reported that electrostatic charges are also responsible for problems in blend uniformity. The net positive or negative tendency of dry powders to become charged electrostatically is called chargeability.

There is no standard instrument for the measurement of the chargeability of dry powders (see AAPS PharmSciTech 2006, 7, Article 103). Triboelectric charge is commonly reported on a charge-to-mass basis since net charge and mass can be easily measured. However, triboelectric and induction charging are more closely related to the surface area of a particle rather to volume or mass. Then, it is well known that the surface area of the particles plays a key role in dry powder chargeability, and that therefore the particles having a higher specific surface area can hold a greater charge.

The authors of the present invention have surprisingly found that, although showing a highly similar mean particle size diameter (by volume) as compared with the crystals with needle-like shape obtained by the prior art processes (i.e. D[4,3] of about 180 μm), the crystals of nepafenac with plate-like shape of the present invention exhibit a more reduced specific surface area and, consequently, a reduced chargeability. Namely, the crystals of nepafenac with plate-like shape of the present invention exhibit a specific surface area of less than 0.800 m²/g, preferably of less than 0.780 m²/g, more preferably of less than 0.760 m²/g, even more preferably of less than 0.740 m²/g, and yet even more preferably of less than 0.720 m²/g.

The nepafenac crystals with plate-like shape of the invention have a particle size distribution in which approximately 10% of the total volume comprises particles having a diameter of approximately 80 μm or below, preferably of approximately 50 μm or below, and more preferably of approximately 40 μm or below; approximately 50% of the total volume comprises particles having a diameter of approximately 400 μm or below, preferably of approximately 300 μM or below, and more preferably of approximately 200 μm or below; and approximately 90% of the total volume comprises particles having a diameter of approximately 1000 μm or below, preferably of approximately 700 μm or below, and more preferably of approximately 500 μm or below.

In another aspect, the present invention provides an inventive process for preparing the nepafenac crystals with plate-like shape of the invention. In particular, the process for preparing nepafenac with plate-like shape of the invention comprises crystallizing nepafenac in a mixture of 2-propanol with up to 40% of water, preferably with between 0.1-40% of water, more preferably with between 1-39% of water, even more preferably with between 5-35% of water, and yet even more preferably with between 10-30% of water.

It has to be noted that the process of the invention above not only has been proved to provide nepafenac with unexpected morphology, i.e. plate-like shape, unexpected specific surface area and low electrostatic characteristics, but also has been proved to show unexpected results.

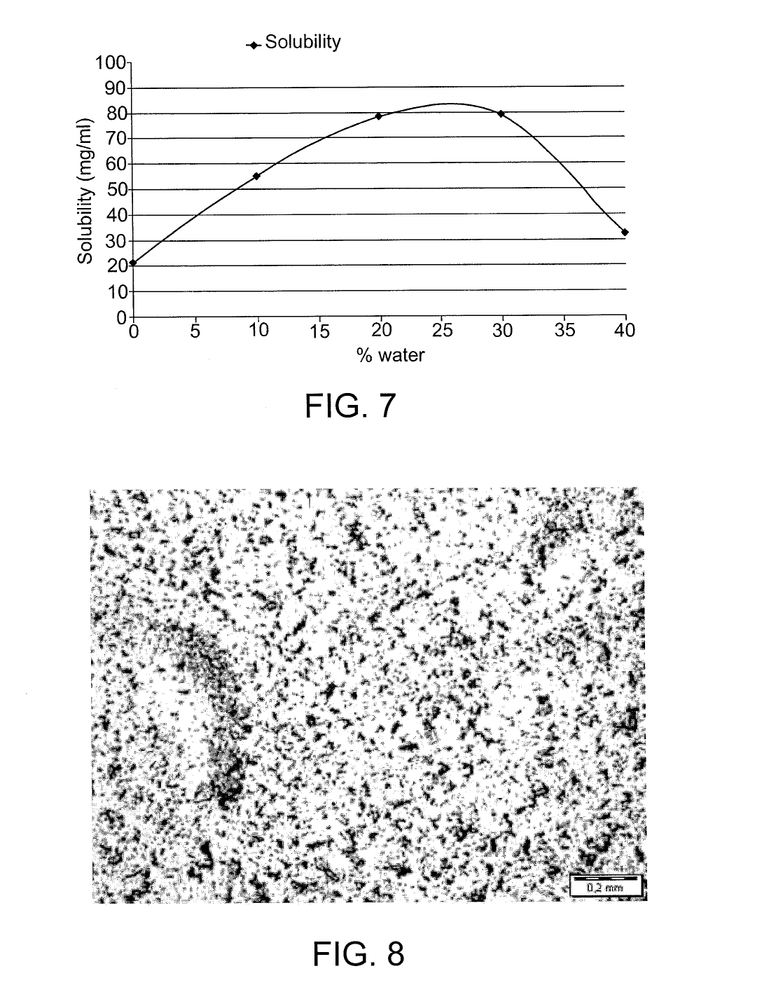

In this regard, nepafenac shows a low solubility profile and its purification process by means of crystallization requires the use of high volumes of an alcohol solvent such as 2-propanol. The present inventors have calculated the solubility profile at reflux temperature of nepafenac, and have confirmed that nepafenac, at reflux temperature, is sparingly soluble in 2-propanol (i.e. solubility=21 mg/mL) and slightly soluble in water (i.e. solubility=0.7 mg/mL). Surprisingly, it has been observed that nepafenac is more soluble in mixtures of 2-propanol with up to 40% of water at reflux temperature (i.e. solubility=30-80 mg/mL). See FIG. 7.

Thus, the present inventors have found that the combination of 2-propanol, a solvent which moderately dissolves nepafenac, with up to 40% of water, a low efficient solvent for nepafenac, surprisingly provides a solvent which is useful for dissolving and crystallizing nepafenac. In addition, the nepafenac obtained by this process shows an unexpected morphology, i.e. plate-like shape, with reduced electrostatic characteristics.

Further, the process of the invention above is suitable for industrial implementation.

In another aspect, the present invention relates to the use of the nepafenac crystals with plate-like shape of the invention for preparing a pharmaceutical composition of nepafenac.

In another further aspect, the present invention relates to the use of the nepafenac crystals with plate-like shape of the invention as starting material for preparing nepafenac crystals with small size. Since the nepafenac crystals with plate-like shape show a reduced electrostatic nature, the process of reducing their size by conventional mechanical methods such as milling is also easier and safer.

In another further aspect, the present invention relates to nepafenac crystals with small size as obtained from reducing the size of the nepafenac crystals with plate-like shape of the invention (See FIG. 8). In this regard, should be noted that it is well known that properties of milled solids can be influenced by the morphology of the starting material used for the milling (Eur. J. Pharm. Sci. 2006, 27, 19-26).

The term “crystals with small size” as used herein is intended to denote a material fanned of small crystals, typically nepafenac crystals having D₉₀ particle size of less than about 150 μm, typically less than about 100 μm more typically less than about 80 μm, even more typically less than about 40 μm, and yet even more typically less than about 20 μm.

Additionally, the present inventors have found that the said nepafenac crystals with small size as obtained from reducing the size of the nepafenac crystals with plate-like shape of the invention surprisingly have improved flowability properties, as compared with the nepafenac crystals with small size as obtained from reducing the size of the nepafenac crystals with needle-like shape of the prior art processes.

Flowability affects the ease with which the material is handled during processing into a pharmaceutical product. Namely, when flowability is very poor, problems occur with handling and processing during formulating. The flowability of nepafenac can be measured using the Hausner ratio, which is a value calculated by dividing the tapped bulk density of nepafenac by the freely settled bulk density of nepafenac. The freely settled bulk density is calculated by pouring a known weight of material into a measuring cylinder and recording the volume. The tapped density is calculated by tapping the cylinder against a surface for a specified number of times and recording again the new volume. See Henry H. Hausner, “Friction Conditions in a Mass of Metal Powders”, Int. J. Powder Metall. Vol. 3, 1967, pp 7-13.

A low Hausner ratio indicates a high flowability. In this regard, it is generally accepted that a Hausner ratio equal to or higher than 1.46 indicates a very poor flowing material, which is rarely acceptable for manufacturing purposes. Therefore, a Hausner ratio less than 1.46 indicates an acceptable flowing material.

The authors of the present invention have found that the nepafenac crystals with small size, as obtained from reducing the size of the nepafenac crystals with needle-like shape of the prior art processes, have a non-desirable very, very poor flowability (i.e. having a Hausner ratio equal to about 1.79, see Example 7). On the other hand, it has been surprisingly found that small size crystals, as obtained from reducing the size of the nepafenac crystals with plate-like shape of the present invention, have a Hausner ratio of less than 1.46 (i.e. 1.43. See Example 8) thus indicating an acceptable flowing material. Since both types of nepafenac crystals with small size have comparable particle size distributions, the improved flowability properties of the nepafenac crystals with small size, as obtained from reducing the size of the nepafenac crystals with plate-like shape of the present invention, were completely unexpected.

In another further aspect, the present invention provides small size crystals of nepafenac prepared from nepafenac crystals with plate-like shape which show improved flowability characteristics (i.e. having a Hausner ratio less than 1.46) and which are therefore acceptable for manufacturing purposes.

The small size crystals of nepafenac prepared from nepafenac crystals with plate-like shape of the present invention have an improved flowability character and thus are better handled and processed during the formulation of the product. Consequently, the small size crystals of nepafenac prepared from nepafenac crystals with plate-like shape of the invention are more suitable for pharmaceutical formulation use.

Measurement of Hausner ratio is well-known in the art and is described, for example, by Mersmann; Crystallization Technology Handbook (A. Mersmann, ed., 2^{nd} Ed., Marcel Dekker). In the present invention, the bulk and tapped densities for each sample of nepafenac were determined using a TP-TD1 tapped densitometer from Pharma Test. The Hausner ratio of the nepafenac sample was calculated by dividing the tapped bulk density by the bulk density.

In another further aspect, the present invention relates to a process for preparing the said crystals of nepafenac having a small size and improved flowability which are prepared from nepafenac crystals with plate-like shape, said process comprising reducing the particle size of nepafenac crystals with plate-like shape.

The reduction of particle size may be achieved via any conventional mechanical process of reducing the size of particles (i.e. mechanical comminution) which includes any one or more of cutting, chipping, grinding, crushing, milling, micronizing, and trituration. Other alternative and/or supplementary methods which entail particle size reduction may be used, such as spray-drying or crystallizing under controlled conditions.

In this regard, according to Pharm. Dev. Technol. 2004, 9, 1-13, the most common way to produce a drug in small particle size is the comminution of previously formed larger particles using milling processes such as jet milling, pearl-ball milling, or high-pressure homogenization. However, in this reference it is described that the mechanical comminution is a mainly uncontrolled and disadvantageous process. The high energy input affects the surface properties, and consequently the bulk properties, of the resulting product. The high energy input can also cause a disruption of the crystal lattice on the particle surface and the creation of defects, such as the formation of amorphous regions which result in a peak widening in powder X-ray diffraction. Namely, a mechanically micronized powder with a thermodynamically activated surface shows a decreased powder flow. Furthermore, mechanical comminution generally results in a broad particle size distribution and heterogeneous particle shapes. Thus, in this reference it is described that due to the disadvantages of milling processes, drug particle engineering techniques like spray-drying, which enable the production of a drug directly in the required particle size, represent an interesting alternative. Since nepafenac is formulated as a suspension for ophthalmic use, a homogeneous particle shape, a reduced particle size and a narrow distribution of particle size should be required, and consequently, taking into account Pharm. Dev. Technol. 2004, 9, 1-13, a spray-dried nepafenac should be anticipated as the most preferred product with small size intended for pharmaceutical use in order to avoid the common disadvantages of mechanically milled compounds.

However, the present inventors have also found that crystals of nepafenac with a small size obtained from spray-drying methods show undesirable characteristics as compared with nepafenac crystals with small size as obtained by mechanical comminution and hence the latter are more desirable for pharmaceutical formulation.

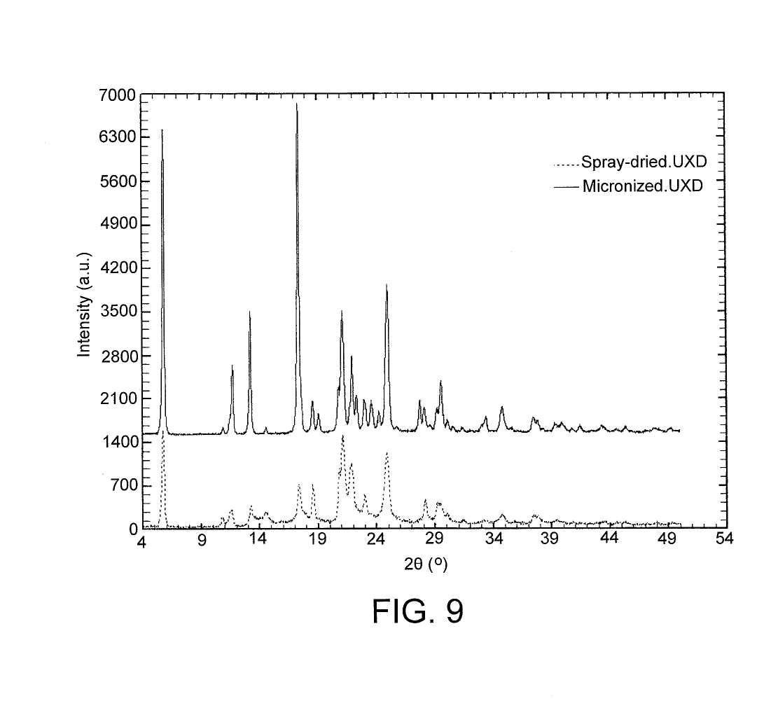

In this respect, the inventors have found that crystals of nepafenac with a small size obtained by spray-drying a solution of nepafenac have a sphericity ratio of about 0.82 whereas crystals of nepafenac with a small size obtained by reducing the size of nepafenac by conventional mechanical processes, e.g. micronizing, show a sphericity ratio of about 1.0, thus indicating that particle shape is closer to spherical particles (See Example 11). These values indicate that crystals of nepafenac with a small size obtained by comminution surprisingly show a homogeneous particle shape, and better flow properties than the nepafenac crystals obtained by spray-drying. Also, these sphericity values indicate that the shape of said crystals of nepafenac with a small size obtained by spray-drying is more irregular and heterogeneous than the shape of the crystals of nepafenac with small size obtained by conventional mechanical reduction processes since the particle shape of the latter is closer to spherical particles. The higher spherical properties of the nepafenac crystals with small particle size obtained by comminution represents a relevant advantage for nepafenac pharmaceutical formulation, since nepafenac is formulated as a suspension and is applied by the topical ocular route, and consequently the said crystals of nepafenac are less potentially cornea damaging (i.e. the more the particle deviate from sphere, the more abrasive will be the formulation for ophtalmic use). Additionally, the nepafenac with small particle size obtained by mechanical comminution (e.g. micronization) shows a smaller particle size than nepafenac obtained by spray drying. Also, the nepafenac obtained by comminution (e.g. micronization) shows narrower peaks by powder X-ray diffraction as compared with the nepafenac obtained by spray-drying (See FIG. 9), thus indicating a higher crystallinity. Further, the said nepafenac crystals with small size and improved sphericity obtained by comminution maybe suitable for pharmaceutical formulation.

One of the most common ways of expressing the degree of irregularity of the particles is by means of the sphericity factor (ψ_{w}), which is generally defined as the ratio between the surface area of a sphere having the same volume as the particle and the surface area of the particle:

$${Ψ}_{W} = \frac{{d}_{V}^{2}}{{d}_{S}^{2}}$$

where dᵥ and dₛ are the equivalent volume and surface diameter, respectively (Part. Part. Syst. Charact. 1996, 13, 368-373).

Because sphericity factors are not calculated for each particle but for an assembly of particles, they have to be based on an average size. Thus, the sphericity factor will then be given by the following equation:

$${Ψ}_{W} = \frac{6}{{S}_{W} \cdot ρ \cdot {D}_{MVS}}$$

where S_{w} is the powder specific surface area, ρ is the particle density and D_{MVS} is the surface area mean diameter, also referred to as the mean volume-surface, the Sauter diameter or D[3,2]. The Sauter diameter is defined as the diameter of a sphere that has the same volume/surface area ratio as the particle of interest. It is important to note that the accuracy of the equation above does not depend on any assumptions, being limited only by experimental conditions.

A sphericity factor of 1.0 describes a perfect sphere with the greatest ease of flow (Encapsulated and powdered foods, CRC Press 2005). The more the particles deviate from spheres (i.e. the sphericity factor decreases from 1.0), the stronger the friction and cohesion forces are, which hence results in reduced flowability.

The true density refers to mass of solid material divided by its exact volume without porosity. It can be directly calculated based on the crystal structure of the compound, as determined by X-ray crystallography (see F. M. Richards, P. F. Lindley, Determination of the density of solids, International Tables for Crystallography, Springer, 2006). Therefore, any crystal shape of a same polymorphic form will show the same density value. It can also be experimentally measured using a pycnometer, if the crystal structure is not available. Calculated values can be also obtained by the Immirzi and Perini prediction method, which has been shown to predict the true density for APIs with a very low average percent error, specially in the range of density values between 1.2 and 1.5 (see Int. J. Pharm. 2008, 355, 231-237).

True density of nepafenac was experimentally found to be 1.33 g/cm³. The experimental value is very closed to that calculated with the Immirzi and Perini prediction method (1.29 g/cm³).

Consequently, the sphericity factor value for nepafenac obtained by spray-drying is substantially lower than that for the product obtained by mechanical comminution, even after taking into account that both values may contain some experimental errors associated with the measures of the density, particle size (mean diameter by surface area to volume) and specific surface area (See Example 11). Therefore, nepafenac with small particle size obtained by mechanical comminution (e.g. micronization) shows better flow properties than nepafenac obtained by spray-drying, i.e. more homogeneous particle shape, improved sphericity, improved flowability, reduced abrasive properties, improved particle size, and improved crystallinity.

Thus, in another aspect, the present invention provides nepafenac with small particle size suitable for pharmaceutical use, characterized by a sphericity factor of more than 0.90, preferably of more than 0.95, more preferably of more than 0.98, and even more preferably of about 1.0.

In another further aspect, the invention provides nepafenac with small particle size suitable for pharmaceutical use, characterized in that the said nepafenac with small particle size has been prepared by comminution (i.e. mechanical method of reducing the particle size).

In yet another aspect, the invention provides a process for preparing the nepafenac with small particle size suitable for pharmaceutical use of the invention, said process comprising (i) providing nepafenac crystals, and (ii) reducing the size of nepafenac crystals by conventional mechanical reduction processes.

In a preferred embodiment, the nepafenac crystals of step (i) of the process above are the nepafenac crystals with plate-like shape of the invention. Therefore, the nepafenac obtained shows improved flowability, i.e. improved Hausner ratio, and improved sphericity and is more suitable for pharmaceutical formulation and use.

The reducing the size of nepafenac crystals by conventional mechanical reduction processes of step (ii) of the process above can comprise any conventional mechanical process of reducing the size of particles which includes any one or more of cutting, chipping, grinding, crushing, milling, micronizing, and trituration. As alternative the reduction of particle size of nepafenac crystals is carried out by crystallizing under controlled conditions.

In an embodiment, the present invention provides a process for preparing crystals of nepafenac having a small size, said process comprising reducing by mechanical reduction processes the size of nepafenac crystals with plate-like shape. Preferably, the mechanical reduction processes comprises micronizing.

When considering the kind of process to be used for reducing the size of crystals of nepafenac, spray-drying must be avoided in view of the defective nepafenac particle sphericity achieved.

The nepafenac crystals having a small size of the invention have a particle size distribution in which approximately 10% of the total volume comprises particles having a diameter of approximately 40 μm or below, preferably of approximately 20 μm or below, more preferably of approximately 10 μm or below, even more preferably of approximately 5 μm or below, and yet even more preferably of approximately 2 μm or below.

The nepafenac crystals having a small size of the invention have a particle size distribution in which approximately 50% of the total volume comprises particles having a diameter of approximately 100 μm or below, preferably of approximately 50 μm or below, more preferably of approximately 30 μm or below, even more preferably of approximately 15 μm or below, and yet even more preferably of approximately 7 μm or below.

The nepafenac crystals having a small size of the invention have a particle size distribution in which approximately 90% of the total volume comprises particles having a diameter of approximately 150 μm or below, preferably of approximately 100 μm or below, more preferably of approximately 80 μm or below, even more preferably of approximately 40 μm or below, and yet even more preferably of approximately 20 μm or below.

The nepafenac crystals with small size of the invention are particularly useful as starting material for preparing a pharmaceutical composition of nepafenac.

Pharmaceutical formulation that comprises the nepafenac crystals having either plate-like shape or small particle size and optionally at least an additional pharmaceutically acceptable excipient or carrier, is another preferred embodiment of present invention. Particularly, suspension formulations for ophthalmic use comprising nepafenac crystals having s sphericity factor of more than 0.90 are preferred.

Moreover, the use of the nepafenac crystals having either plate-like shape or small particle size, as anti-inflammatory drug is also envisaged in present invention.

Another embodiment of the invention consists in a method of prevention and/or treatment of inflammatory diseases comprising the administration to a subject of a therapeutically effective amount or dose of a formulation comprising nepafenac crystals having either plate-like shape or small particle size.

### SPECIFIC EXAMPLES

The following examples further illustrate the invention but, of course, should not be construed as in any way limiting its scope.

### General Experimental Conditions

### Particle Size Distribution Method:

The particle size for nepafenac was measured using a Malvern Mastersizer S particle size analyzer with an MS1-Small Volume Sample Dispersion Unit stirred cell. A 300RF mm lens and a beam length of 2.4 mm were used. Samples for analysis were prepared by wetting a weighed amount of nepafenac (approximately 50 mg) with 0.5 mL of a 1% solution of Igepal CA-630 in deionized water, and dispersing in 20 mL of deionized water. After sonication for 1 minute, the suspension was delivered drop-wise to the previously background and corrected measuring cell filled with dispersant (deionized water) until the obscuration reached the desired level. Volume distributions were obtained for three times. After completing the measurements, the sample cell was emptied and cleaned, refilled with suspending medium, and the sampling procedure repeated again. For characterization, the values of D₁₀, D₅₀ and D₉₀ (by volume), D[4,3] (mean diameter by volume) and D[3,2] (mean diameter by surface area to volume, or Sauter diameter) were specifically listed, each one being the mean of the six values available for each characterization parameter.

The notation Dₓ means that X % of the particles have a diameter less than a specified diameter D. Thus a D₉₀ [or D(v, 0.9)] of 100 μm means that 90% of the particles have a diameter less than 100 μm.

### Optical Microscopy:

A solid sample (containing nepafenac crystals with either needle-like shape or plate-like shape) or an immersion oil suspension (containing nepafenac crystals with small size) was mounted on a slide and analyzed using an Olympus BX41 microscope. The micrographs were taken at 40× magnification.

The aspect ratio of crystals was obtained from micrographs of a batch of crystal. Micrographs were processed with ImageJ 1.42q software. Length and width of at least 100 representative crystals (i.e., having an area greater than 500 μm² for a 40× magnification image) was measured, and the aspect ratio of each crystal was calculated by dividing the crystal length by the crystal width. The average aspect ratio for each batch was determined by dividing the sum of crystal aspect ratios by the number of crystals measured.

### Specific Surface Area Method:

The BET (Brunauer, Emmett and Teller) specific surface area for nepafenac was measured using a Micromeritics™ GEMINI V equipment (GEMINI CONFIRM V2.00 Software™). The sample for analysis was degassed at 30° C. for 10 minutes and at 140° C. for one hour. The determination of the adsorption of N₂ at 77 K was measured for relative pressures in the range of 0.02 to 0.2 for a weighed amount of nepafenac (i.e., approximately 0.5 g).

### Density:

Density of nepafenac samples was determined at 25° C. using a 50 mL glass pycnometer. A pre-weighted amount of about 0.5 to 1 g of nepafenac was introduced in the pycnometer, and the volume was filled with n-heptane, where nepafenac is practically insoluble at the working temperature. The density of the nepafenac sample (ρₛ) can be determined from the known density of n-heptane (ρ_{H}: 0.685 g/cm³), the weight of the pycnometer filled only with n-heptane (W_{H}⁰, the weight of the filled pycnometer containing both nepafenac and n-heptane (W_{S+H}), and the weight of nepafenac (W_{S}):

$${ρ}_{S} = \frac{{W}_{S} \cdot {ρ}_{H}}{{W}_{H}^{0} + {W}_{S} - {W}_{S + H}}$$

Density of nepafenac samples was determined for three times, being the listed result the mean of the three values available for each sample.

Density of nepafenac was also calculated by the Immirzi and Perini method, using the following formula:

$$ρ = \frac{1.645 \cdot M}{{V}_{S}}$$

where M is the molecular weight of nepafenac (254.28 g/mol) and V_{S} is the calculated crystal volume for a single molecule (angstrom³/molecule) which is expressed by the following equation:

$${V}_{S} = {\sum }_{j} {m}_{j} \cdot {v}_{j}$$

where mⱼ is the relative stoichiometric multiplicities and vⱼ is the volume increments of elements or ions (angstrom³). For nepafenac, V_{S} is calculated using the following volume increments (vⱼ):

| | No. of | | |
|---|---|---|---|
| | elements | | |
| | in nepafenac | | |
| Element | structure (mⱼ) | vⱼ | mⱼ · vⱼ |
| —H | 14 | 6.9 | 96.6 |
| >C═ | 2 | 13.7 | 27.4 |
| >C< | 1 | 11.0 | 11.0 |
| ═O | 2 | 14.0 | 28.0 |
| >N— | 2 | 7.2 | 14.4 |
| benzene ring | 2 | 75.2 | 150.4 |
| N—H . . . O hydrogen bond (—CONH₂) | 1 | −2.8 | −2.8 |

| | |
|---|---|
| ${V}_{S} = {\sum }_{j} {m}_{j} \cdot {v}_{j}$ | 325.0 |

and, therefore:

$$ρ = \frac{1.645 \cdot 254.28}{325.0} = 1.287 g \text{/} {cm}^{3}$$

### Comparative Example 1 and Examples 1 to 4

### Preparation of Different Crystals of 2-amino-3-benzoylbenzeneacetamide (i.e. Nepafenac) Under Different Crystallization Conditions

General procedure: Nepafenac was dissolved under magnetical stirring and at reflux in a solvent (See Table 1). The solution was cooled to room temperature under stirring. The solid was filtered and dried. The different conditions and the obtained results are described in Table 1 below.

TABLE 1

| | | | Solvent | Solubility | Crystal |
|---|---|---|---|---|---|
| Example | Quantity | Solvent | quantity | (mg/mL) | shape |
| Com- | 657 mg | 2-propanol | 31 mL | 21.2 | Needle |
| parative | | | | | |
| Example 1 | | | | | |
| 1 | 580 mg | 2- | 10.5 mL | 55.2 | Plate |
| | | propanol:water | | | |
| | | 90:10 | | | |
| 2 | 589 mg | 2- | 7.5 mL | 78.6 | Plate |
| | | propanol:water | | | |
| | | 80:20 | | | |
| 3 | 597 mg | 2- | 7.5 mL | 79.6 | Plate |
| | | propanol:water | | | |
| | | 70:30 | | | |
| 4 | 619 mg | 2- | 19 mL | 32.6 | Plate |
| | | propanol:water | | | |
| | | 60:40 | | | |

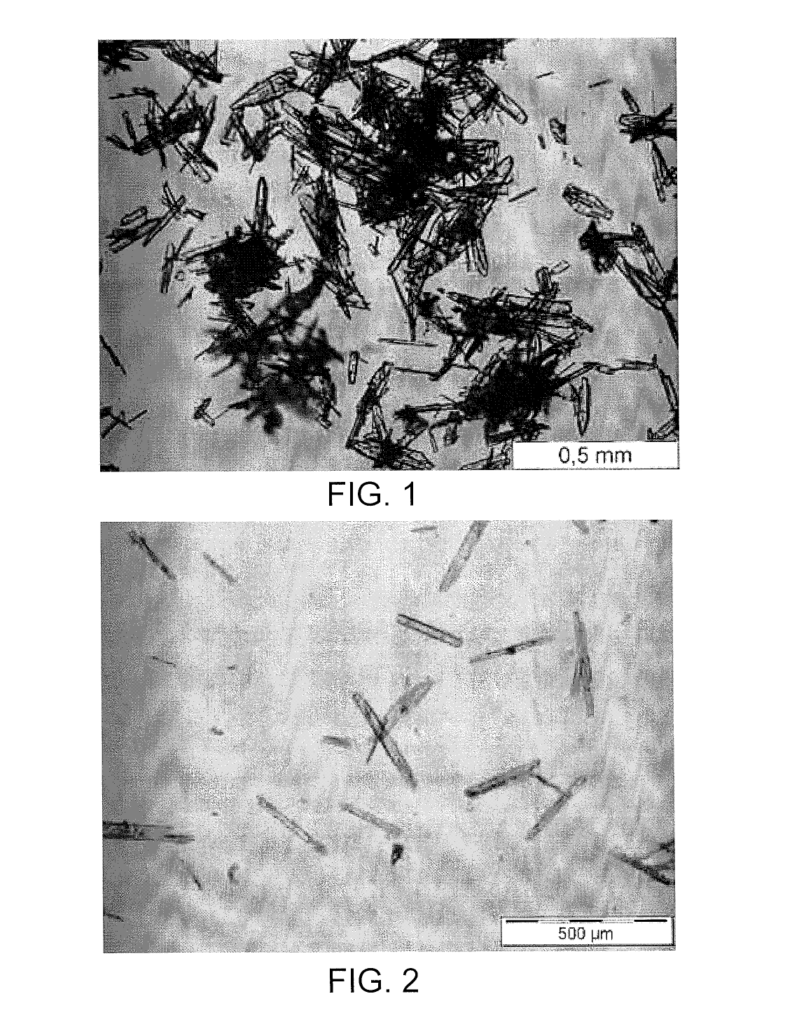

The nepafenac obtained in Comparative Example 1 showed crystals of needle-like shape (FIG. 1). High chargeability was observed when handling these crystals with a metallic material.

The nepafenac obtained in Examples 1-4 showed crystals of plate-like shape (FIG. 4). Reduced chargeability was observed as compared with the needle-like crystals when handling these crystals with a metallic material. Aspect ratio: 3.04 (Example 1).

### Comparative Example 2

### Preparation of 2-amino-3-benzoylbenzeneacetamide (i.e., Nepafenac) Crystals of Needle-Like Shape

A solution of 55.4 g of nepafenac in 3200 mL of hot 2-propanol was allowed to cool to room temperature. The solid was filtered and dried under vacuum at 60° C. yielding 45.9 g of nepafenac as a yellow solid.

Optical Microscopy: Needle-like crystals (FIG. 2). Aspect ratio: 5.67.

### Comparative Example 3

### Preparation of 2-amino-3-benzoylbenzeneacetamide (i.e., Nepafenac) Crystals of Needle-Like Shape

22.50 g of 2-amino-3-benzoylbenzeneacetamide were dissolved in 1100 mL of 2-propanol at reflux temperature. The solution was cooled to 20° C. under stirring in 3 hours. The solid was filtered and dried under vacuum at 40° C. yielding 20.7 g of nepafenac as a yellow solid.

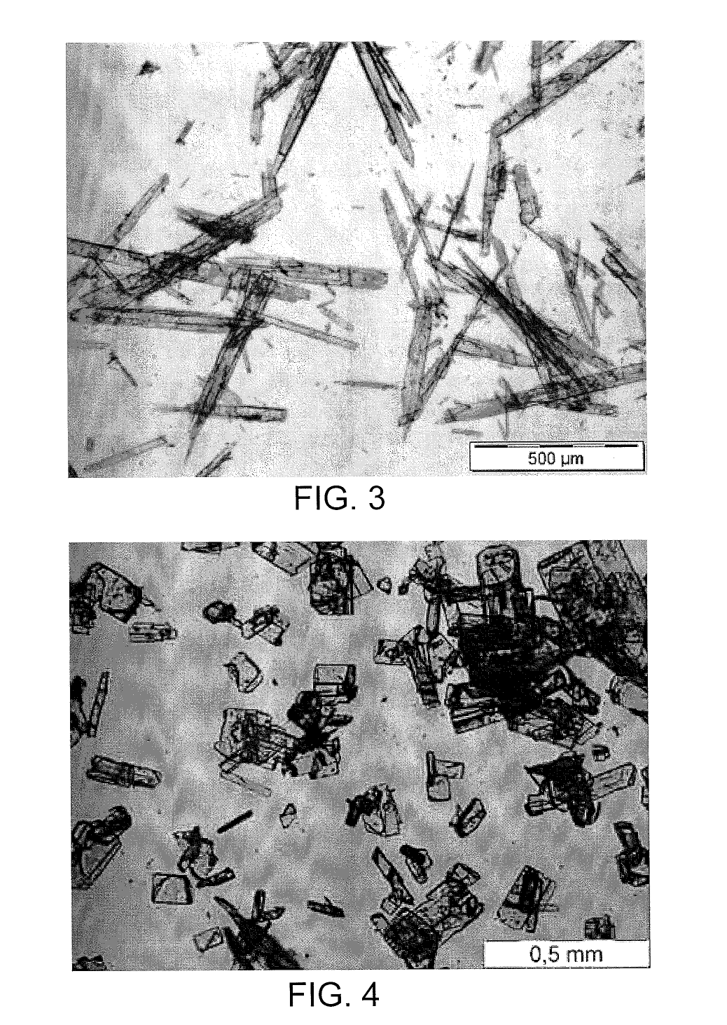

Particle Size Distribution: D₁₀: 23.6 μm, D₅₀: 85.8 μm, D₉₀: 491.1 μm; D[4,3]: 178.4 Specific Surface Area (BET): 0.8256±0.0270 m²/g. Optical Microscopy: Needle-like crystals (FIG. 3).

### Example 5

### Preparation of 2-amino-3-benzoylbenzeneacetamide (i.e., Nepafenac) Crystals of Plate-Like Shape

310 g of Nepafenac were dissolved under mechanical stirring in 7.5 L of a 2-propanol:water mixture 90:10 at reflux temperature. The solution was cooled to 20° C. and stirred at this temperature. The solid was filtered and dried under vacuum at 60° C. until constant weight.

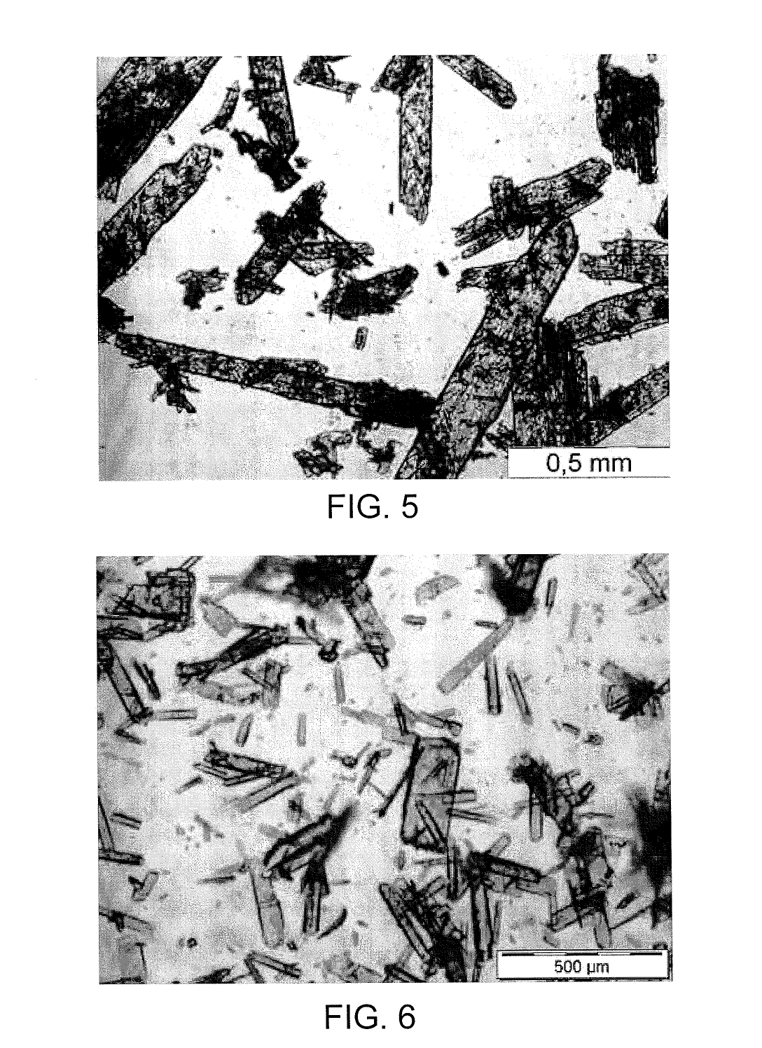

The obtained nepafenac showed crystals of plate-like shape (FIG. 5). Reduced chargeability was observed as compared with the needle-like crystals when handling these crystals with a metallic material.

### Example 6

### Preparation of 2-amino-3-benzoylbenzeneacetamide (i.e., Nepafenac) Crystals of Plate-Like Shape

22.50 g of 2-amino-3-benzoylbenzeneacetamide were dissolved in 600 mL of a 2 propanol:water mixture 90:10 at reflux temperature. The solution was cooled to 20° C. under stirring in 3 hours. The solid was filtered and dried under vacuum at 40° C. yielding 20.2 g of nepafenac as a yellow solid.

Particle Size Distribution: D₁₀: 30.5 μm, D₅₀: 144.9 μm, D₉₀: 401.8 μm; D[4,3]: 185.0 μm. Specific Surface Area (BET): 0.7046±0.0272 m²/g. Optical Microscopy: plate-like crystals (FIG. 6).

### Examples 7-8

### Preparation of 2-amino-3-benzoylbenzeneacetamide (i.e., Nepafenac) Crystals with Small Size

Nepafenac in form of crystals with needle-like shape as obtained in Comparative Example 3, and in form of crystals with plate-like shape as obtained in Example 6 were slowly introduced in a RINA-JET Turbo-micronizer with controlled parameters (Pventuri: 5 bar; Pmilling: 3 bar), and were micronized.

The resultant products were analyzed, and the results obtained are shown in Table 2.

TABLE 2

| | Starting | | | | Hausner |
|---|---|---|---|---|---|
| Example | material | D₁₀ | D₅₀ | D₉₀ | ratio |
| 7 | Needle-like | 1.3 μm | 6.1 μm | 17.1 μm | 1.79 |
| 8 | Plate-like | 1.2 μm | 5.8 μm | 18.9 μm | 1.43 |

The nepafenac with plate-like shape of Example 8 showed a Powder X-ray diffractogram similar to FIG. 9.

### Example 9

### Preparation of 2-amino-3-benzoylbenzeneacetamide (i.e., Nepafenac) Crystals with Small Size

Three different samples of nepafenac obtained according to Example 5 were introduced in a RINA-JET Turbo-micronizer with controlled parameters (Pventuri: 5 bar; Pmilling: 3 bar), and were micronized.

The resultant products were analysed, and the results obtained are shown in Table 3. The results were confirmed with microscopic observation.

TABLE 3

| Samples | D₁₀ | D₅₀ | D₉₀ |
|---|---|---|---|
| Batch 1 | 6.1 | 21.4 | 64.3 |
| Batch 2 | 4.8 | 19.8 | 66.5 |
| Batch 3 | 4.4 | 14.9 | 45.3 |

Analytical data for micronized nepafenac: m.p.=183.7-184.8° C. Optical Microscopy: see FIG. 8.

### Example 10

### Preparation of 2-amino-3-benzoylbenzeneacetamide (i.e., Nepafenac) Crystals with Small Size by Spray-Drying

4.01 g of nepafenac was dissolved in 750 mL of acetone. The solution was filtered and spray dried using a Buchi B290 spray dryer. The dried nepafenac was recovered.

The following parameters were used: inlet temperature (actual reading)=85° C., outlet temperature (actual reading)=65° C., aspirator=100% (equivalent to approximately 35 m³/hour), nitrogen flow=30 mm (equivalent to approximately 360 L/hour). The peristaltic pump to feed the product solution was set to 10% (equivalent to approximately 3.5 mL/min).

Particle Size Distribution: D₁₀: 2.2 μm, D₅₀: 12.1 μm, D₉₀: 27.5 μm; D[3,2]: 2.7 μm. Specific Surface Area (BET): 2.03410.0074 m²/g. XRD: See FIG. 9.

### Example 11

Sphericity factors for some batches of nepafenac are calculated and summarized in Table 4.

TABLE 4

| | | | D_{MVS} | | |
|---|---|---|---|---|---|
| Example | Type | ρ (g/cm³) | (μm) | S_{w} (m²/g) | Ψ_{w} |
| 8 | Micronized | 1.33 | 2.5 | 1.7686 ± 0.0242 | 1.0 |
| 10 | Spray-drying | 1.33 | 2.7 | 2.0341 ± 0.0074 | 0.82 |

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. Nepafenac crystals with plate-like shape.

2. The nepafenac crystals according to claim 1, characterized by having an length/width aspect ratio of approximately 5.6 or below.

3. The nepafenac crystals according to claim 1, characterized by having a specific surface area of less than 0.800 m²/g.

4. The nepafenac crystals according to claim 2, characterized by having a specific surface area of less than 0.800 m²/g.

5. The nepafenac crystals according to claim 1, characterized in that said crystals have a particle size distribution in which approximately 90% of the total volume comprises particles have a diameter of approximately 1000 μm or below.

6. The nepafenac crystals according to claim 2, characterized in that said crystals have a particle size distribution in which approximately 90% of the total volume comprises particles have a diameter of approximately 1000 μm or below.

7. The nepafenac crystals according to claim 3, characterized in that said crystals have a particle size distribution in which approximately 90% of the total volume comprises particles have a diameter of approximately 1000 μm or below.

8. A process for preparing the nepafenac crystals with plate-like shape of claim 1, said process comprising crystallizing nepafenac in a mixture of 2-propanol with up to 40% of water.

9. A process for preparing the nepafenac crystals with plate-like shape of claim 2, said process comprising crystallizing nepafenac in a mixture of 2-propanol with up to 40% of water.

10. A process for preparing the nepafenac crystals with plate-like shape of claim 3, said process comprising crystallizing nepafenac in a mixture of 2-propanol with up to 40% of water.

11. A process for preparing the nepafenac crystals with plate-like shape of claim 4, said process comprising crystallizing nepafenac in a mixture of 2-propanol with up to 40% of water.

12. A process for preparing the nepafenac crystals with plate-like shape of claim 5, said process comprising crystallizing nepafenac in a mixture of 2-propanol with up to 40% of water.

13. A process for preparing the nepafenac crystals with plate-like shape of claim 6, said process comprising crystallizing nepafenac in a mixture of 2-propanol with up to 40% of water.

14. A process for preparing the nepafenac crystals with plate-like shape of claim 7, said process comprising crystallizing nepafenac in a mixture of 2-propanol with up to 40% of water.

15. Nepafenac crystals having a small size suitable for pharmaceutical use, characterized by having a Hausner ratio of less than 1.46.

16. Nepafenac crystals having a small size suitable for pharmaceutical use, characterized by a sphericity factor of more than 0.90.

17. The nepafenac crystals of claim 15, characterized in that said crystals have a particle size distribution in which approximately 90% of the total volume comprises particles having a diameter of approximately 150 μm or below.

18. The nepafenac crystals of claim 16, characterized in that said crystals have a particle size distribution in which approximately 90% of the total volume comprises particles having a diameter of approximately 150 μm or below.

19. A process for preparing crystals of nepafenac with small size, said process comprising (i) providing nepafenac crystals, and (ii) reducing the size of nepafenac crystals by conventional mechanical size reduction processes.

20. The process according to claim 19, characterized in that the reduction of particle size of nepafenac crystals is carried out by one or more of cutting, chipping, grinding, crushing, milling, micronizing, or trituration.

21. The process according to claim 19, characterized in that the reduction of particle size of nepafenac crystals is carried out by crystallizing under controlled conditions.

22. The process according to claim 19, characterized in that the nepafenac crystals of step (i) are the nepafenac crystals with plate-like shape of claim 1.

23. A pharmaceutical formulation comprising the nepafenac crystals of claim 1 and optionally at least an additional pharmaceutically acceptable excipient or carrier.

24. A pharmaceutical formulation comprising the nepafenac crystals of claim 15 and optionally at least an additional pharmaceutically acceptable excipient or carrier.

25. A method of treating inflammatory diseases comprising administering to a subject a therapeutically effective amount of the pharmaceutical formulation of claim 24.
